(19) United States
(12) Patent Application Publication
(10) Pub. No.: US 2005/0059861 A1
(43) Pub. Date: Mar. 17, 2005

(54) Flexible tube of endoscope and method for manufacturing the same

(75) Inventor: Takehiro Nishiie, Tokyo (JP)

Correspondence Address: SCULLY SCOTT MURPHY & PRESSER, PC, 400 GARDEN CITY PLAZA, GARDEN CITY, NY, 11530, US

(73) Assignee: Olympus Corporation, Tokyo (JP)

(21) Appl. No.: 10/939,733

(22) Filed: Sep. 13, 2004

(30) Foreign Application Priority Data
Sep. 11, 2003 (JP) 2003-320286

(51) Int. Cl. A61B 1/005

(52) U.S. Cl. 600/144; 600/140

(57) ABSTRACT

A first flexibility-varying portion is disposed in an outer sheath of a flexible tube having a distal end and a base end. The thickness of the outer sheath in the distal end side of the first flexibility-varying portion is varied from the thickness in the base end side, and the flexibility of the flexible tube in the distal end side is varied from the flexibility in the base end side. A second flexibility-varying portion is disposed in the outer sheath, at a location different from that of the first flexibility-varying portion in a longitudinal direction of the flexible tube. The second flexibility-varying portion is disposed substantially without varying the thickness of the outer sheath in the longitudinal direction of the flexible tube, and the flexibility of the flexible tube in the distal end side is varied from the flexibility in the base end side.

This application claims benefit of Japanese Application No. 2003-320286 filed on Sep. 11, 2003, the contents of which are incorporated by this reference.

### BACKGROUND OF THE INVENTION

1. Field of the Invention

The present invention relates to a flexible tube of an endoscope used in the fields of medical care, industries, and the like and to a method for manufacturing the same.

2. Description of the Related Art

In recent years, endoscopes have been widely used for the intracavitary inspection and diagnosis, the inspection of the inside of a plant, and the like in the medical field and the industrial field. In general, a flexible tube of an endoscope has the configuration in which the outer surface of a helical tube is covered with a net-shaped tube and, furthermore, the outer surface of the net-shaped tube is covered with an outer sheath resin.

With respect to these known flexible tubes of endoscopes, the compounding ratio of the outer sheath resin is varied, or an outer sheath resin is formed into the shape of a taper, as disclosed in Japanese Unexamined Patent Application Publication No. 2001-190494, for example.

### SUMMARY OF THE INVENTION

A flexible tube of an endoscope according to the present invention is provided with a first flexibility-varying portion disposed in an outer sheath of a flexible tube having a distal end and a base end, while the thickness of the outer sheath in the distal end side of the first flexibility-varying portion is varied from the thickness in the base end side and the flexibility of the flexible tube in the distal end side is varied from the flexibility in the base end side. A second flexibility-varying portion is disposed in the outer sheath, at a location different from that of the first flexibility-varying portion in a longitudinal direction of the flexible tube, while the second flexibility-varying portion is disposed substantially without varying the thickness of the outer sheath in the longitudinal direction of the flexible tube and the flexibility of the flexible tube in the distal end side is varied from the flexibility in the base end side.

In a method for manufacturing a flexible tube of an endoscope according to the present invention, a first covering layer is formed on a member formed substantially in the shape of a long-length tube having a distal end and a base end, while the first covering layer includes a flexibility-varying portion which varies the flexibility of the flexible tube in a longitudinal direction substantially without varying the thickness. Subsequently, a second covering layer is formed on the outside of the first covering layer, while the second covering layer includes a flexibility-varying portion which varies the flexibility of the flexible tube in the longitudinal direction by varying the thickness from the distal end side toward the base end side of the flexible tube. Thereafter, the first covering layer and the second covering layer are brought into intimate contact with each other.

According to the flexible tube of the endoscope of the present invention and the method for manufacturing the same, such excellent effects are exerted that the manufacture is performed easily, a large flexibility difference between the softest portion and the hardest portion can be ensured and, in addition, the gradient of the flexibility variation can be made smooth.

The above features and advantages of the invention will become more clearly understood from the following description referring to the accompanying drawings.

### BRIEF DESCRIPTION OF THE DRAWINGS

FIG. 1 is a schematic configuration diagram showing the entire electronic endoscope, according to a first embodiment of the present invention.

FIG. 2 is a schematic configuration diagram showing the internal structure of a flexible tube, according to the first embodiment of the present invention.

FIG. 3A is a sectional view of the flexible tube, according to the first embodiment of the present invention.

FIG. 3B is an explanatory diagram of the flexibility of the flexible tube, according to the first embodiment of the present invention.

FIG. 4A is an explanatory diagram of a step of applying a covering of an outer sheath resin serving as the second layer of the flexible tube, according to the first embodiment of the present invention.

FIG. 4B is an explanatory diagram of the state in which a heat-shrinkable tube in the state shown in FIG. 4A has been peeled off.

FIG. 5A is an explanatory diagram of the insertion of the stepped flexible tube shown in FIG. 4B into a grinder.

FIG. 5B is an explanatory diagram showing the state of the stepped flexible tube shown in FIG. 4B after being ground with the grinder.

FIG. 6A is a sectional view of a flexible tube, according to a second embodiment of the present invention.

FIG. 6B is an explanatory diagram of the flexibility of the flexible tube, according to the second embodiment of the present invention.

FIG. 7A is a sectional view of a flexible tube, according to a third embodiment of the present invention.

FIG. 7B is an explanatory diagram of the flexibility of the flexible tube, according to the third embodiment of the present invention.

### DETAILED DESCRIPTION OF THE PREFERRED EMBODIMENTS

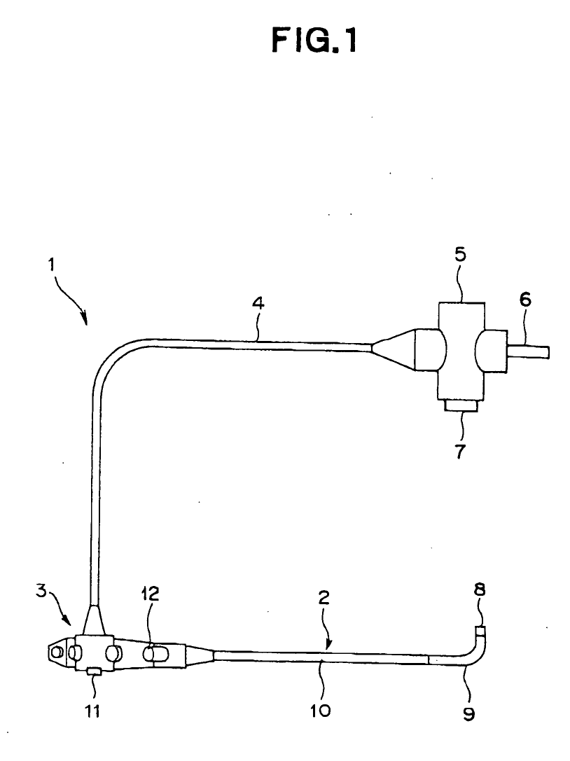

A first embodiment of the present invention will be described below with reference to FIG. 1 to FIG. 5B.

In FIG. 1, reference numeral 1 denotes an electronic endoscope adopted in the first embodiment. The electronic endoscope 1 includes a slender insertion portion 2, a control portion 3 which is connected to a proximal end side of the insertion portion 2 and which is grasped by a surgeon to perform various operations, and a universal cord 4 which is disposed extending from the control portion 3.

At the other end of the universal cord 4, a connector portion 5 connected to a light source and a camera control unit (hereafter abbreviated as “CCU”), although not shown in the drawing, is disposed. In this case, a light guide connector 6 is connected to the light source, and a camera connector 7 is connected to the CCU.

The insertion portion 2 includes a flexible tube 10 having flexibility, joined to the control portion 3, a bending portion 9 joined to the distal end of the flexible tube 10, and a distal end 8 joined to the distal end of the bending portion 9. On the other hand, the control portion 3 is provided with, for example, a bending control lever 11 for controlling the bending of the bending portion 9 in the vertical and horizontal direction and a therapeutic instrument insertion hole 12 for inserting a therapeutic instrument, e.g., forceps.

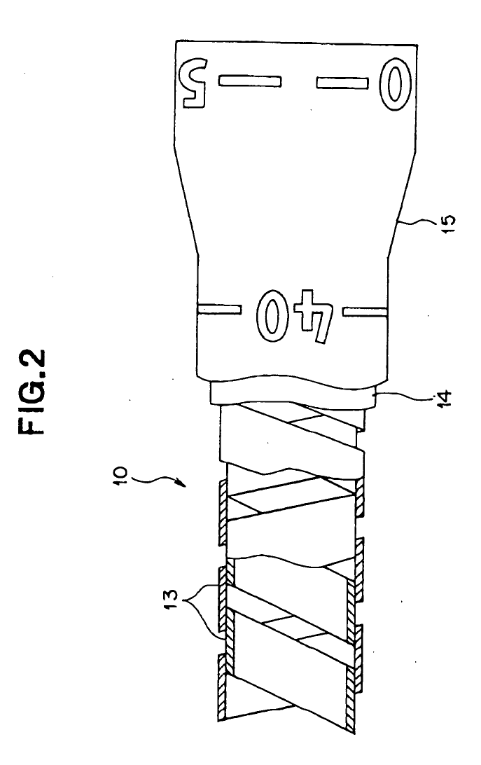

As shown in FIG. 2, the flexible tube 10 is primarily composed of a helical tube 13 in which two pieces of metal band are wound helically, a net-shaped tube 14 formed into the shape of a net covering the helical tube 13, and an outer sheath resin 15 covering the outside of the net-shaped tube 14 in that order from the inside. The outer sheath resin 15 Is formed into the shape of a taper at some midpoint of the flexible tube 10, while the taper has a diameter increasing from the distal end side toward the base end side, as is clear from the appearance thereof.

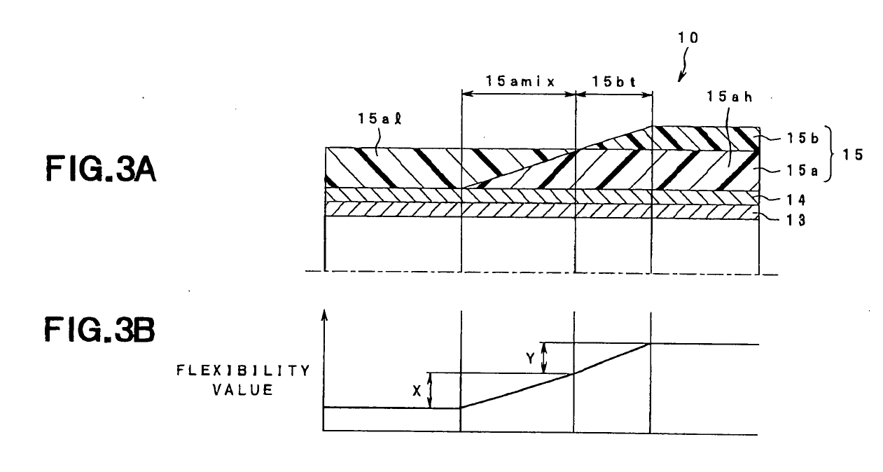

As shown in FIG. 3A, the outer sheath resin 15 of the flexible tube 10 is composed of a plurality of layers, incidentally, is composed of two layers in the first embodiment. An outer sheath resin 15a, which is the first layer in the net-shaped tube 14 side of the outer sheath resin 15, is formed by compounding a soft resin as a first resin (distal end side) and a hard resin as a second resin (base end side) (in FIG. 3A, reference numeral 15al denotes a soft resin portion of the outer sheath resin 15a serving as the first layer and reference numeral 15ah denotes a hard resin portion of the outer sheath resin 15a serving as the first layer).

Specifically, in the outer sheath resin 15a serving as the first layer, the portion from the distal end of the flexible tube to the position at a distance of about 200 mm from the distal end (an index of about 30 when expressed in the index of flexible tube, where the index of flexible tube refers to a distance from the distal end of an endoscope) is formed of the soft resin portion 15al. With respect to the outer sheath resin 15a serving as the first layer, in the portion from the position at a distance of about 200 mm from the distal end of the flexible tube to the position at a distance of about 300 mm (an index of about 40 in terms of index of flexible tube), the soft resin portion 15al is gradually replaced with the hard resin portion 15ah, and this portion constitutes a resin-hardness-varying portion 15a mix serving as the second flexibility-varying portion. Here, with respect to the resin materials for the outer sheath resin 15a serving as the first layer, for example, the soft resin portion 15al is composed of an ester-based resin, and the hard resin portion 15ah is composed of the same-ester-based but harder resin.

In an outer sheath resin 15b serving as the second layer which is an outermost layer, while the outer sheath resin 15b is disposed outside the outer sheath resin 15a serving as the first layer of the outer sheath resin 15, a taper portion 15bt serving as a first flexibility-varying portion is disposed. The taper portion 15bt has a wall thickness gradually increasing from the position substantially following the base end side of the resin-hardness-varying portion 15a mix of the outer sheath resin 15a serving as the first layer toward the base end side is disposed. Here, the same ester-based resin as that used for the soft resin portion 15al of the outer sheath resin 15a serving as the first layer is used as the resin material for the outer sheath resin 15b serving as the second layer.

Steps for manufacturing the above-described flexible tube 10 will be described below.

In the first step, as in known flexible tubes, a covering of the outer sheath resin 15a serving as the first layer composed of a soft resin and a hard resin is applied. As described above, in the outer sheath resin 15a serving as the first layer, the portion from the distal end of the flexible tube to the position at a distance of about 200 mm from the distal end is formed of the soft resin portion 15al. In the portion from this position at a distance of about 200 mm from the distal end of the flexible tube to the position at a distance of about 300 mm, the soft resin portion 15al is gradually replaced with the hard resin portion 15ah, and this portion constitutes a resin-hardness-varying portion 15a mix.

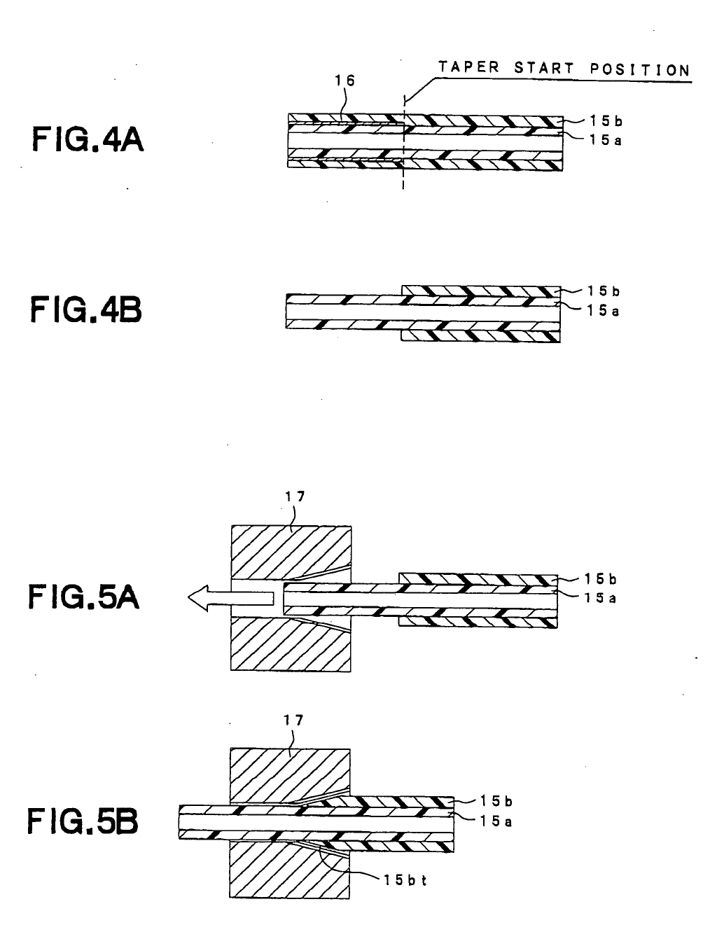

In the second step, the outer sheath resin 15a serving as the first layer is covered with a heat-shrinkable tube 16, up to the location in the neighborhood of the position at which the replacement of the soft resin portion 15al with the hard resin portion 15ah is completed (in the neighborhood of the position indicated by an index of about 40), that is, up to the start position of the taper portion 15bt at which the resin-hardness-varying portion 15a mix terminates, and under this condition, a covering of the outer sheath resin 15b serving as the second layer is applied (FIG. 4A).

In the third step 5 the outer sheath resin 15b serving as the second layer located in the side nearer to the distal end than is the taper start position is peeled off together with the heat-shrinkable tube 16, so that a stepped flexible tube shown in FIG. 4B is prepared.

In the forth step, as shown in FIG. 5A and FIG. 5B, the taper portion 15bt is formed from the stepped flexible tube prepared in the third step by the use of a grinder 17. The grinding may be performed either by rotation of the grindstone side of the grinder 17 or by rotation of the stepped flexible tube. Alternatively, a high-heat mold or the like which partially fuses the taper portion 15bt may be used in place of the grinder 17.

In the fifth step, the net-shaped tube 14 and the outer sheath resin 15a serving as the first layer are joined by heat adhesion, and likewise, the outer sheath resin 15a serving as the first layer and the outer sheath resin 15b serving as the second layer are joined by heat adhesion. At this time, it is desirable that the heat adhesion is performed at 180° C. to 220° C. for 10 to 15 minutes, for example. However, the heat-adhesion condition is not limited to this as long as an optimum condition is set based on the melting points and the heat resistance of the outer sheath resin 15a serving as the first layer and the outer sheath resin 15b serving as the second layer.

In the sixth step, the white line ink and the top coat are applied as in known flexible tubes.

With respect to the thus prepared flexible tube 10 according to the first embodiment of the present invention, by dividing the manufacturing process into a step of ensuring the adhesion between the net-shaped tube 14 and the outer sheath resin 15a serving as the first layer and between the outer sheath resin 15a serving as the first layer and the outer sheath resin 15b serving as the second layer (in particular, the fifth step), a step of applying the coverings of the outer sheath resins 15a and 15b in which it is essential only that the outer diameters of the small diameter portion and the large diameter portion of the flexible tube 10 are satisfied (in particular, the first and the second steps), and a step of forming the taper portion 15bt in which it is essential only that the length and the start position of the taper portion 15bt of the outer sheath resin 15b serving as the second layer are satisfied (in particular, the third and the fourth steps), determination of each optimum condition becomes easy, and the flexible tube 10 is manufactured optimally and easily.

Since no adhesive is required in the fifth step of joining by the heat adhesion, fluctuations in the adhesion due to variations in the adhesive are reduced. In particular, in the neighborhood of the start position of the taper portion 15bt, no adhesive extends off the portion where the outer sheath resin 15b serving as the second layer is disposed, nor occurs, conversely, shortage of the adhesive in the neighborhood of the start position of the taper portion 15bt.

With respect to the flexible tube 10 according to the first embodiment of the present invention, as shown in FIG. 3B, initially, smooth flexibility variation as in known flexible tubes is achieved by the resin-hardness-varying portion 15a mix of the outer sheath resin 15a serving as the first layer (a portion X in FIG. 3B). Subsequently, the flexibility variation due to the taper portion 15bt of the outer sheath resin 15b serving as the second layer is achieved substantially following the resin-hardness-varying portion 15a mix (a portion Y in FIG. 3B). In this manner, in the entire flexible tube 10, the large flexibility variation can be achieved with a gentle, long gradient.

In the above-described flexible tube 10, a larger load is applied to the outer sheath resin 15b serving as the second layer when the flexible tube 10 is bended repeatedly. This is because larger distortion due to the bending occurs in the outer sheath resin 15b serving as the second layer located farther from the center line of the flexible tube 10. However, the bending resistance can be improved by using a sort resin for the outer sheath resin 15b serving as the second layer.

Further, various specifications are made for the desired flexible tube 10. However, the flexible tube 10 according to the first embodiment of the present invention can be flexibly complied with various specifications required for the flexibility variation by combining the shapes of the outer sheath resin 15a serving as the first layer, the outer sheath resin 15b serving as the second layer, and the taper portion 15bt and a compounding ratio of each resin. For example, with respect to the taper portion 15bt of the outer sheath resin 15b serving as the second layer, a softer resin provides gentler flexibility variation even when the shapes of the taper portions are the same. Conversely, a harder resin provides a larger amount of flexibility variation. With respect to the taper portion 15bt, even when the resins are the same, if the gradient of the taper shape were gentler, the flexibility variation would become gentler, and if the gradient of the taper shape were steeper, the amount of flexibility variation would become larger.

The flexible tube 10 according to the first embodiment of the present invention is described with reference to the example in which the eater-based resin is used as the resin material for the soft resin portion 15al of the outer sheath resin 15a serving as the first layer, and the same-ester-based but harder resin is used for the hard resin portion 15ah. The description is made with reference to the example in which the same ester-based resin as that used for the soft resin portion 15al of the outer sheath resin 15a serving as the first layer is used as the resin material for the outer sheath resin 15b serving as the second layer. However, these resin materials are not limited thereto, and resins of, for example, ester-based, olefin-based, styrene-based, and amide-based may be used alone or after blending for the outer sheath resins serving as respective layers in accordance with the required flexibility.

The flexible tube 10 according to the first embodiment of the present invention has a configuration in which the resin-hardness-varying portion 15a mix of the outer sheath resin 15a serving as the first layer is substantially followed by the taper portion 15bt of the outer sheath resin 15b serving as the second layer. However, these are not necessarily continuous. For example, some distance may be provided between the resin-hardness-varying portion 15a mix of the outer sheath resin 15a serving as the first layer and the taper portion 15bt of the outer sheath resin 15b serving as the second layer, or the two may overlap with each other to some extent.

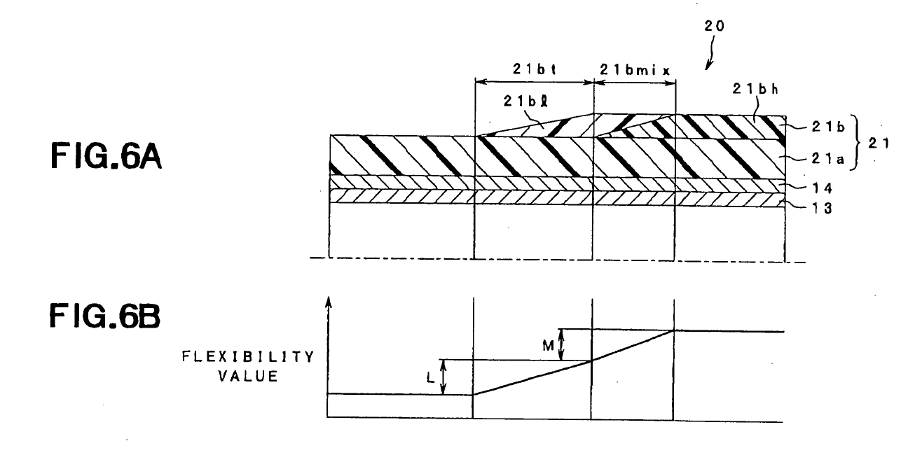

A second embodiment of the present invention will be described below with reference to FIG. 6A and FIG. 6B.

In the second embodiment, in contrast to the above-described first embodiment, both the resin-hardness-varying portion serving as the flexibility-varying portion to vary the flexibility, and the taper portion are disposed in the outer sheath resin serving as the second layer. The other configurations are similar to those in the first embodiment and, therefore, the explanation thereof will not be provided.

That is, in FIG. 6A, reference numeral 20 denotes a flexible tube. The flexible tube 20 is primarily composed of a helical tube 13, a net-shaped tube 14 formed to have the shape of a net covering the helical tube 13, and an outer sheath resin 21 covering the outside of the net-shaped tube 14 in that order from the inside. The outer sheath resin 21 is formed into the shape of a taper at some midpoint of the flexible tube 20, while the taper has a wall thickness increasing from the distal end side toward the base end side, as is clear from the appearance thereof.

The outer sheath resin 21 of the flexible tube 20 is composed of a plurality of layers, incidentally, is composed of two layers in the second embodiment. An outer sheath resin 21a, which is the first layer in the net-shaped tube 14 side of the outer sheath resin 21, is formed of one type of soft resin, for example.

In an outer sheath resin 21b serving as the second layer which is disposed outside the outer sheath resin 21a serving as the first layer of the outer sheath resin 21, a taper portion 21bt serving as a first flexibility-varying portion is disposed in the distal end side while having a wall thickness gradually increasing toward the base end side. The outer sheath resin 21b serving as the second layer is formed by compounding a soft resin as a first resin (distal end side) and a hard resin as a second resin (base end side) (in FIG. 6A, reference numeral 21bl denotes a soft resin portion of the outer sheath resin 21b serving as the second layer and reference numeral 21bh denotes a hard resin portion of the outer sheath resin 21b serving as the second layer).

Specifically, the outer sheath resin 21b serving as the second layer is formed of the soft resin portion 21bl up to the position in the neighborhood of the position where the taper portion 21bt terminates. In the outer sheath resin 21b serving as the second layer, a resin-hardness-varying portion 21b mix serving as the second flexibility-varying portion is formed, in which the soft resin portion 21bl is gradually replaced with the hard resin portion 21bh from the position in the neighborhood of the position where the taper portion 21bt of the soft resin portion 21bl terminates.

Here, an ester-based resin, for example, is used as the resin material for the outer sheath resin 21a serving as the first layer. With respect to the rein material for the outer sheath resin 21b serving as the second layer, for example, the same ester-based resin as that used for the outer sheath resin 21a serving as the first layer is used for the soft resin portion 21bl, and the same-ester-based but harder resin is used for the hard resin portion 21bh. However, selection of these resin materials is not limited thereto, and resins of, for example, ester-based, olefin-based, styrene-based, and amide-based may be used alone or after blending for the outer sheath resins serving as respective layers in accordance with the required flexibility.

With respect to the thus prepared flexible tube 20 according to the second embodiment of the present invention, as in the above-described first embodiment, by dividing the manufacturing process into a step of ensuring the adhesion between the net-shaped tube 14 and the outer sheath resin 21a serving as the first layer and between the outer sheath resin 21a serving as the first layer and the outer sheath resin 21b serving as the second layer, a step of applying the coverings of the outer sheath resins 21a and 21b in which it is essential only that the outer diameters of the small diameter portion and the large diameter portion of the flexible tube 20 are satisfied, and a step of forming the taper portion 21bt in which it is essential only that the length and the start position of the taper portion 21bt of the outer sheath resin 21b serving as the second layer are satisfied, determination of each optimum condition becomes easy, and the flexible tube 20 can be manufactured optimally and easily.

Since no adhesive is required in the joining by the heat adhesion as in the above-described first embodiment, fluctuations in the adhesion due to variations in the adhesive are reduced. In particular, in the neighborhood of the start position of the taper portion 21bt, no adhesive extends off the portion where the outer sheath resin 21b serving as the second layer is disposed, nor occurs, conversely, shortage of the adhesive in the neighborhood of the start position of the taper portion 21bt.

Further, with respect to the flexible tube 20 according to the second embodiment of the present invention, as shown in FIG. 6B, initially, smooth flexibility variation as in known flexible tubes is achieved by the taper portion 21bt of the outer sheath resin 21b serving as the second layer (a portion L in FIG. 6B). Subsequently, the flexibility variation due to the resin-hardness-varying portion 21b mix of the outer sheath resin 21b serving as the second layer is achieved substantially following the taper portion 21bt (a portion M in FIG. 6B). In this manner. in the entire flexible tube 20, the large flexibility variation can be achieved with a gentle, long gradient.

In the above-described flexible tube 20, a larger load is applied to the outer sheath resin 21b serving as the second layer when the flexible tube 20 is bended repeatedly. This is because larger distortion due to the bending occurs in the outer sheath resin 21b serving as the second layer located farther from the center line of the flexible tube 20. However, the bending resistance can be improved by using a soft resin for the outer sheath resin 21b serving as the second layer.

Further, various specifications are made for the desired flexible tube 20. However, the flexible tube 20 according to the second embodiment of the present invention can be flexibly complied with various specifications required for the flexibility variation by combining the shapes of the outer sheath resin 21a serving as the first layer, the outer sheath resin 21b serving as the second layer, and the taper portion 21bt and a compounding ratio of each resin.

The flexible tube 20 according to the second embodiment of the present invention has a configuration in which the taper portion 21bt of the outer sheath resin 21b serving as the second layer is substantially followed by the resin-hardness-varying portion 21b mix. However, these are not necessarily continuous. For example, some distance may be provided between the taper portion 21bt and the resin-hardness-varying portion 21b mix, or the two may overlap with each other to some extent.

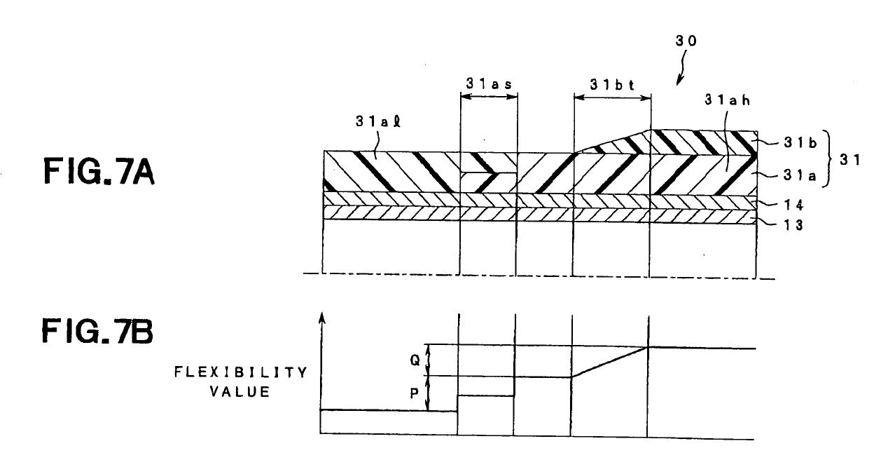

A third embodiment of the present invention will be described below with reference to FIG. 7A and FIG. 7B.

In the third embodiment, in contrast to the above-described first embodiment, the outer sheath resin serving as the first layer is composed of a plurality of tube-shaped resins different in flexibility. The other configurations are similar to those in the first embodiment and, therefore, the explanation thereof will not be provided.

That is, in FIG. 7A, reference numeral 30 denotes a flexible tube. The flexible tube 30 is primarily composed of a helical tube 13, a net-shaped tube 14 formed to have the shape of a net covering the helical tube 13, and an outer sheath resin 31 covering the outside of the net-shaped tube 14 in that order from the inside. The outer sheath resin 31 is formed into the shape of a taper at some midpoint of the flexible tube 30, while the taper has a wall thickness gradually increasing from the distal end side toward the base end side, as is clear from the appearance thereof.

The outer sheath resin 31 of the flexible tube 30 is composed of a plurality of layers, incidentally, is composed of two layers in the third embodiment. With respect to an outer sheath resin 31a, which is the first layer in the net-shaped tube 14 side of the outer sheath resin 31, the distal end side is formed of a soft tube resin 31al as a first resin, and the base end side is formed of a hard tube resin 31ah as a second resin. The end surface in the base end side of the soft tube resin 31al is formed to have a large inner diameter, and the end surface in the distal end side of the hard tube resin 31ah is formed to have a small outer diameter. In the outer sheath resin 31a serving as the first layer, the base end side of the soft tube resin 31al and the distal end side of the hard tube resin 31ah are engaged to constitute a engage portion 31 as serving as a second flexibility-varying portion (a resin-hardness-varying portion).

In an outer sheath resin 31b serving as the second layer which is disposed outside the outer sheath resin 31a serving as the first layer of the outer sheath resin 31, a taper portion 31bt (a first flexibility-varying portion) is disposed in the distal end side while having a wall thickness gradually increasing from the start point located at a predetermined distance from the engage portion 31 as of the outer sheath resin 31a serving as the first layer toward the base end side.

Here, for example, an eater-based resin is used as the resin material for the soft tube resin 31al of the outer sheath resin 31a serving as the first layer, and the same-ester-based but harder resin is used for the hard tube resin 31ah. For example, the same ester-based resin as that used for the soft tube resin 31al of the outer sheath resin 31a serving as the first layer is used as the resin material for the outer sheath resin 31b serving as the second layer. However, selection of these resin materials is not limited thereto, and resins of, for example, ester-based, olefin-based, styrene-based, and amide-based may be used alone or after blending for the outer sheath resins serving as respective layers in accordance with the required flexibility.

With respect to the thus prepared flexible tube 30 according to the third embodiment of the present invention, as in the above-described first embodiment, by dividing the manufacturing process into a step of ensuring the adhesion between the net-shaped tube 14 and the outer sheath resin 31a serving as the first layer and between the outer sheath resin 31a serving as the first layer and the outer sheath resin 31b serving as the second layer, a step of applying the coverings of the outer sheath resins 31a and 31b in which it is essential only that the outer diameters of the small diameter portion and the large diameter portion of the flexible tube 30 are satisfied, and a step of forming the taper portion 31bt in which it is essential only that the length and the start position of the taper portion 31bt of the outer sheath resin 31b serving as the second layer are satisfied, determination of each optimum condition becomes easy, and the flexible tube 30 can be manufactured optimally and easily.

Since no adhesive is required in the joining by the heat adhesion as in the above-described first embodiment, fluctuations in the adhesion due to variations in the adhesive are reduced. In particular, in the neighborhood of the start position of the taper portion 31bt, no adhesive extends off the portion where the outer sheath resin 31b serving as the second layer is disposed, nor occurs, conversely, shortage of the adhesive in the neighborhood of the start position of the taper portion 31bt.

With respect to the flexible tube 30 according to the third embodiment of the present invention, as shown in FIG. 7B, initially, small flexibility variation is achieved by the engage portion 31as of the outer sheath resin 31a serving as the first layer. subsequently, small stepwise flexibility variation is further achieved in between the engage portion 31as and the taper portion 31bt (a portion P in FIG. 7B). In the following taper portion 31bt, smooth flexibility variation is achieved (a portion Q in FIG. 7B). In this manner, in the entire flexible tube 30, the large flexibility variation can be achieved stepwise with a gentle, long gradient.

In the above-described flexible tube 30, a larger load is applied to the outer sheath resin 31b serving as the second layer when the flexible tube 30 is bended repeatedly. This is because larger distortion due to the bending occurs in the outer sheath resin 31b serving as the second layer located farther from the center line of the flexible tube 30. However, the bending resistance can be improved by using a soft resin for the outer sheath resin 31b serving as the second layer.

Further, various specifications are made for the desired flexible tube 30. However, the flexible tube 30 according to the third embodiment of the present invention can be flexibly complied with various specifications required for the flexibility variation by combining the shapes of the outer sheath resin 31a serving as the first layer, the outer sheath resin 31b serving as the second layer, and the taper portion 31bt, the length and the shape of the engage portion 31as of the outer sheath resin 31a serving as the first layer, and a compounding ratio of each resin.

In the flexible tube 30 according to the third embodiment of the present invention, a predetermined distance is provided between the engage portion 31as of the outer sheath resin 31a serving as the first layer and the taper portion 31bt of the outer sheath resin 31b serving as the second layer. However, the engage portion 31as and the taper portion 31bt may be disposed in a substantially continuous manner, or the two may be disposed in the locations overlapping with each other.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

1. A flexible tube of an endoscope, comprising:
a first flexibility-varying portion disposed in an outer sheath of a flexible tube having a distal end and a base end, while the first flexibility-varying portion is disposed in order that the thickness of the outer sheath in the distal end side of the first flexibility-varying portion is varied from the thickness in the base end side and that the flexibility of the flexible tube in the distal end side is varied from the flexibility in the base end side; and
a second flexibility-varying portion disposed in the outer sheath, at the location different from that of the first flexibility-varying portion in a longitudinal direction of the flexible tube, while the second flexibility-varying portion is disposed in order that the thickness of the outer sheath is substantially not varied in the longitudinal direction of the flexible tube and that the flexibility of the flexible tube in the distal end side is varied from the flexibility in the base end side.

2. The flexible tube of an endoscope according to claim 1,
wherein the outer sheath is composed of a plurality of covering layers, and
wherein the first flexibility-varying portion is composed of a taper portion which is disposed in an outermost covering layer and which makes the thickness of the outermost covering increase from the distal end side toward the base end side.

3. The flexible tube of an endoscope according to claim 1, wherein the second flexibility-varying portion is composed of a resin-hardness-varying portion in which a first resin is replaced with a second resin harder than the first resin from the distal end side toward the base end side of the outer sheath.

4. The flexible tube of an endoscope according to claim 2, wherein the second flexibility-varying portion is composed of a resin-hardness-varying portion in which a first resin is replaced with a second resin harder than the first resin from the distal end side toward the base end side of the outer sheath.

5. The flexible tube of an endoscope according to claim 3, wherein the resin-hardness-varying portion has a configuration in which the first resin disposed in the shape of a tube in the distal end side and the second resin disposed in the shape of a tube in the base end side are joined stepwise to each other.

6. The flexible tube of an endoscope according to claim 4, wherein the resin-hardness-varying portion has a configuration in which the first resin disposed in the shape of a tube in the distal end side and the second resin disposed in the shape of a tube in the base end side are joined stepwise to each other.

7. The flexible tube of an endoscope according to claim 1, wherein the first flexibility-varying portion and the second flexibility-varying portion are disposed in order that the respective sections serving for varying the flexibility of the flexible tube are arranged continuously or with a predetermined distance therebetween.

8. The flexible tube of an endoscope according to claim 2, wherein the first flexibility-varying portion and the second flexibility-varying portion are disposed in order that the respective sections serving for varying the flexibility of the flexible tube are arranged continuously or with a predetermined distance therebetween.

9. The flexible tube of an endoscope according to claim 4, wherein the first flexibility-varying portion and the second flexibility-varying portion are disposed in order that the respective sections serving for varying the flexibility of the flexible tube are arranged continuously or with a predetermined distance therebetween.

10. The flexible tube of an endoscope according to claim 6, wherein the first flexibility-varying portion and the second flexibility-varying portion are disposed in order that the respective sections serving for varying the flexibility of the flexible tube are arranged continuously or with a predetermined distance therebetween.

11. The flexible tube of an endoscope according to claim 1, wherein the first flexibility-varying portion and the second flexibility-varying portion are disposed to overlap with each other in a longitudinal direction of the outer sheath resin.

12. The flexible tube of an endoscope according to claim 2, wherein the first flexibility-varying portion and the second flexibility-varying portion are disposed to overlap with each other in a longitudinal direction of the outer sheath resin.

13. The flexible tube of an endoscope according to claim 4, wherein the first flexibility-varying portion and the second flexibility-varying portion are disposed to overlap with each other in a longitudinal direction of the outer sheath resin.

14. The flexible tube of an endoscope according to claim 6, wherein the first flexibility-varying portion and the second flexibility-varying portion are disposed to overlap with each other in a longitudinal direction of the outer sheath resin.

15. A method for manufacturing a flexible tube of an endoscope, the method comprising the steps of:
forming a first covering layer on a member formed substantially in the shape of an elongated tube having a distal end and a base end, while the first covering layer includes a flexibility-varying portion which varies the flexibility of the flexible tube in a longitudinal direction substantially without varying the thickness;
forming a second covering layer on the outside of the first covering layer, while the second covering layer includes a flexibility-varying portion which varies the flexibility of the flexible tube in the longitudinal direction by varying the thickness from the distal end side toward the base end side of the flexible tube; and
bringing the first covering layer and the second covering layer into intimate contact with each other.

16. The method for manufacturing a flexible tube of an endoscope according to claim 15, wherein the flexibility-varying portion of the second covering layer is formed by disposing a taper portion in order that the thickness of the second covering layer is increased from the distal end side toward the base end side.

17. The method for manufacturing a flexible tube of an endoscope according to claim 16, wherein the second covering layer is disposed as an outermost portion of the outer sheath covering the member formed substantially in the shape of a tube.

18. The method for manufacturing a flexible tube of an endoscope according to claim 15, wherein the flexibility-varying portion of the first covering layer is formed by replacing a first resin with a second resin harder than the first resin from the distal end side toward the base end side of the first covering layer.

19. The method for manufacturing a flexible tube of an endoscope according to claim 17, wherein the taper portion is formed by grinding or fusing.

20. The method for manufacturing a flexible tube of an endoscope according to claim 15, wherein bringing into intimate contact is performed by heat adhesion.
